(19) United States
(12) Patent Application Publication
(10) Pub. No.: US 2007/0071778 A1
(43) Pub. Date: Mar. 29, 2007

(54) Stable microemulsion concentrate for delivery of a bioactive biocide/disinfectant/fungicide/fragrance in an aqueous medium providing sustained release

(75) Inventors: Kolazi S. Narayanan, Wayne, NJ (US); Jayanti Patel, Elmwood Park, NJ (US); John J. Merianos, Middletown, NJ (US); Susan M. Lindstrom, Ramsey, NJ (US); Karen Winkowski, Springfield, NJ (US); Xianbin Liu, Basking Ridge, NJ (US); Donald I. Prettypaul, Englewood, NJ (US)

Correspondence Address: Attn: William J. Davis, Esq.;INTERNATIONAL SPECIALTY PRODUCTS, Legal Department, Building No. 8, 1361 Alps Road, Wayne, NJ, 07470, US

(73) Assignee: ISP INVESTMENTS INC.

(21) Appl. No.: 11/236,888

(22) Filed: Sep. 28, 2005

(51) Int. Cl. A61K 9/14; A61K 33/14; A61K 33/36; A61K 36/13

(52) U.S. Cl. 424/400; 424/770; 424/661; 424/672; 424/486

(57) ABSTRACT

What is described herein is a stable microemulsion concentrate and composition for delivery of an active biocide/disinfectant in an aqueous medium providing sustained release of the active. The composition is effective against both bacteria and fungi.

### BACKGROUND OF THE INVENTION

1. Field of the Invention

This invention relates to microemulsion concentrates, and, more particularly, to a microemulsion concentrate and composition which is microdispersible in water as a clear, solution suitable for effective delivery of an active biocide/disinfectant/fungicide/fragrance to provide sustained release of the active to protect against bacteria and fungi.

2. Description of the Prior Art

Aqueous solutions of iodine in polyvinylpyrrolidone are well known in the art, e.g. a Betadine® solution. However, the iodine loading in such solutions is quite low. Accordingly, it would be advantageous to provide a stable microemulsion for iodine which provides for a high loading of the halogen so that it can be more active against a wide variety of bacteria. Particularly desired are stable microemulsion concentrates for such biocide/disinfectant/fungicide/fragrance ingredients which can form a stable aqueous microemulsion composition upon dilution with water which is effective against both bacteria and fungi.

These and other features and objects of the invention will be made apparent from the following description of the invention.

### SUMMARY OF THE INVENTION

What is described herein is a stable microemulsion concentrate which includes, by wt.

(a) 0.1-20% of a bioactive ingredient, and

(b) 80-99.9% of a water-based polymeric matrix including an alkylated vinyl pyrrolidone copolymer.

### DETAILED DESCRIPTION OF THE INVENTION

In a preferred form of the invention, the stable microemulsion concentrate includes, by wt.

(a) 0.5-10% of a bioactive ingredient, and

(b) 90-99.5% of a polymeric matrix composition of
(i) a C₈-C₁₈ alkyl pyrrolidone, e.g. octyl pyrrolidone,
(ii) an emulsifier, e.g. an anionic or cationic emulsifier, or quat, and
(iii) an aqueous alkylated vinyl pyrrolidone copolymer, e.g. a C₁₆ alkyl PVP, e.g. Ganex® (ISP).

Preferably, (a) is iodine, triclosan or iodopropargyl butyl carbamate (IPC), or a fragrance, or mixtures thereof.

Other suitable biocides/disinfectants which may be used include: oganometallics, iodophor, nitrogen compounds e.g. (Lysol®), organosulfurs, phenolics, chloroisocyanurate, aldehydes e.g. glutaraldehyde, pine oil, and quarternary ammonium compounds e.g. BARQUAT® 42 Z-10, alkyl dimethyl benzyl ammonium saccharinate, and alkyl dimethyl benzyl ammonium chloride.

Suitable anionic emulsifiers (b) (ii) include sodium dodecyl sulfonate or sulfate, sodium laureth sulfonate or sulfate, or sodium dodecyl benzene sulfonate.

In another embodiment of the invention, there is provided a stable microemulsion composition of the active which includes the microemulsion concentrate of the invention and water of dilution.

The invention will now be described in more detail by the following examples.

### EXAMPLE 1

A Stock Solution of a polymer matrix in the form of a microemulsion was prepared as follows: In a 64 oz stoppered glass bottle 483 g of N-octyl pyrrolidone was weighed in and 417 g of an aqueous solution of a 29% sodium lauryl sulfate solution was added. Then 850 g of the Stock Solution was used to dissolve 150 g of Ganex® 516 (ISP) [C₁₆ alpha olefin grafted polyvinyl pyrrolidone having 50% of the vinyl pyrrolidone moiety]. The solid polymer was obtained by evaporation of the commercial product (a 50% solution in isopropyl alcohol). The resulting aqueous Matrix AM-1 composition contained 15% Ganex® copolymer as a microemulsion.

Matrix (AM-1)

| Ingredient | Weight % |
|---|---|
| N-Octyl pyrrolidone | 45.6 |
| Ganex ® copolymer | 15 |
| Sodium lauryl sulfate | 11.4 |
| Water | 28 |
| Total | 100% |

### EXAMPLE 2

### Matrix AM-2

80 g of the Stock Solution was used to dissolve 20 g solid Ganex® 516 copolymer to produce 100 g of the aqueous Matrix AM-2.

### EXAMPLE 3

### Matrix AM-3

90 g of the Stock Solution was used to dissolve 10 g solid Ganex® 516 copolymer to produce 100 g of aqueous Matrix AM-3.

The matrices of Examples 1-3 then were used to prepare concentrates containing bioactive ingredients including iodine, triclosan, and iodo propargyl butyl carbamate (IPBC), and fragrance mixtures.

### Iodine Concentrate and Water of Dilution Compositions

### EXAMPLE 4

10 g of iodine was dissolved in 90 g of Matrix AM-1. The iodine solution was homogeneous and dark brown in color. The resulting concentrate was reduced to a 1% total iodine by diluting 10 g of the concentrate to 100 g with deionized water. The 1% iodine solution was light brown in color with a mild odor of iodine and was non-irritating to the skin. Both concentrate and diluted solutions were stable without separation for a period of at least one month.

The 1% total iodine solution was used to evaluate the biological activity of the composition. The results are shown in Example 16 below in comparison with a commercially equivalent 1% Betadine® solution (PVP-iodine).

### EXAMPLE 5

15 g of iodine was dissolved in 85 g of Matrix AM-1. The iodine solution was homogeneous and dark brown in color. The resulting concentrate was diluted to 1% total iodine by diluting 6.7 g to 100 g with deionized water.

### EXAMPLE 6

Example 4 was repeated using Matrix AM-2 in the place of Matrix AM-1.

### EXAMPLE 7

Example 4 was repeated using Matrix AM-3 in the place of Matrix AM-1.

### Triclosan Concentrate and Water of Dilution Compositions

### EXAMPLE 8

1 g of triclosan was dissolved in 99 g of Matrix AM-1. The resulting triclosan solution was homogeneous and optically clear. This concentrate was diluted with water to 1/10, 1/20 and 1/100 to produce aqueous microemulsions containing 1000 ppm, 500 ppm and 100 ppm of triclosan, respectively. Both concentrate and dilution compositions were optically clear and remained stable without separation for at least one month at room temperature.

### EXAMPLE 9

Example 8 was repeated using 5 g triclosan dissolved in 95 g of Matrix AM-1. The 5% concentrate obtained was a clear, homogeneous solution. This concentrate was diluted to 1/50, 1/100 and 1/500 to produce aqueous microemulsions at 1000 ppm, 500 ppm and 100 ppm of triclosan, respectively. Both concentrate and all dilution compositions were optically clear and remained stable without separation for at least one month at room temperature.

The concentrate was evaluated (See Example 17) for biological activity and sustained release on a gypsum board under saturated conditions with a challenge dose of fungi. The results are shown in comparison with commercial products.

### IPBC Concentrate and Water of Dilution Compositions

### EXAMPLE 10

5 g of IPBC was dissolved in 95 g of Matrix AM-1. The concentrate was a clear, homogeneous solution without separation for at least one month. Dilution to 200 ppm IPBC at 1/250 dilution produced an optically clear microemulsion which was stable without separation for at least one month.

### EXAMPLE 11

Example 10 was repeated using 10 g of IPBC and 90 g of Matrix AM-1. The concentrate obtained was optically clear. This concentrate was used to evaluate its biological efficacy on wood compared to other commercially available compositions. The results showed that the 10% IPBC concentrate was comparable to commercial formulations containing 20% IPBC, indicating substantial biological efficacy.

Accelerated storage stability of the IPBC concentrate at 50° C. for 30 days showed >97% retention of the IPBC activity compared to commercial formulations which had <90% retention of its IPBC (HPLC analysis).

### EXAMPLE 12

Example 11 was repeated using 20 g of IPBC and 80 g of Matrix AM-2. The concentrate was a thixotropic gel. On dilution to 200 ppm at 1/1000 dilution, a stable microemulsion was produced.

### Fragrance Concentrates and Water of Dilution Compositions

### EXAMPLE 13

5 g of perfumed Blooming Mango® Mod-2 (P&G) was dissolved in 95 g of Matrix AM-2 and stirred in a stoppered bottle for 2 hours in an orbital shaker to produce a clear Perfume Concentrate containing 5% Perfume. The concentrate was diluted to 1/10, 1/100, 1/500 and 1/1000 in deionized water. The resulting aqueous Perfume Compositions contained 500 ppm, 100 ppm and 50 ppm Perfume, respectively. The compositions were optically clear as observed over a period of 30 days at room temperature.

### EXAMPLE 14

A body wash composition was prepared as shown in Table 1.

TABLE 1
Body Wash Formulation

| Phase | Ingredient | Wt. % |
|---|---|---|
| A | DI water | 51.15 |
| | Standapol ES-3 | 15.00 |
| B | Mirataine CB | 15.00 |
| | Versene NA | 0.10 |
| | Glycerin | 4.50 |
| C | Germaben II-E | 0.75 |
| D | DI water | 11.25 |
| | NaCl | 2.25 |
| | Total | 100.00% |

Procedure for making body wash.
1. Combine phase A ingredients with stirring. Heat to 45° C.
2. Combine phase B ingredients and heat to 45° C. with stirring.
3. When both phases are uniform, add phase B to phase A with stirring.
4. Add phase C with stirring.
5. Combine phase D with mixing until uniform.
6. Slowly add phase D to batch with stirring.
7. Make up for water loss and stir to room temperature.

Then 1 part of the 5% Perfume Concentrate of Example 13 was added to 99 parts of the Body Wash Formulation. A clear body composition containing 500 ppm of fragrance was obtained. Similarly, 1 part of the 5% Perfume Concentrate added to 499 parts of the Body Wash Formulation provided a clear body wash composition containing 100 ppm of fragrance.

### EXAMPLE 15

The fragrance compositions of Examples 13 and 14 were evaluated for perfume retention with time compared to fragrances diluted in deionized water. Selected dilutions were evaluated by a single panelist for fragrance release over time. One panelist was used for this evaluation. 10 grams of the dilutions were placed into vials label A to E. Two vials were prepared for each sample. One was kept closed (control), while the other was left open and evaluated for the fragrance level with time. The panelist was asked to rank the open vials on a degree of difference using a scale of 1 to 5 as to how different the strength is from the initial strength, control, over time with 1 meaning no change and 5 meaning a large difference. Each sample was evaluated against itself with the control being kept closed and given a score of 1.

The results are shown in Table 2 below.

TABLE 2

| | Example | | | | |
|---|---|---|---|---|---|
| | Concentrate | Concentrate | Concentrate | Concentrate | |
| | at 1:100 | at 1:500 | at 1:100 | at 1:500 | 5,000 ppm |
| | dilution in | dilution in | dilution in | dilution in | fragrance in |
| | water | water | body wash | body wash | DI water |
| Concentrate | 5% | 5% fragrance | 5% fragrance | 5% fragrance | 0.5% |
| Composition | fragrance in | in AM-1 | in AM-1 | in AM-1 | fragrance in |
| | AM-1 | | | | DI water |
| Appearance | Clear | Clear | Clear | Clear | N/A |
| After dilution | Clear | Clear | Clear | Clear | Clear |
| Fragrance | 500 ppm | 100 ppm | 500 ppm | 100 ppm | 0.5% |
| concentration | | | | | |
| after dilution | | | | | |
| Polymer:Fragrance | 3:1 | 3:1 | 3:1 | 3:1 | none |
| ratio | | | | | |

Observation

| | 500 ppm | 100 ppm | 500 ppm | 100 ppm | 0.5% |
|---|---|---|---|---|---|
| Time (hours) | fragrance | fragrance | fragrance | fragrance | fragrance |
| 0.5 | 1.0 | 2.0 | 1.0 | 1.0 | 4.0 |
| 1.0 | 1.0 | 2.5 | 1.0 | 1.0 | 4.0 |
| 1.5 | 2.0 | 2.5 | 1.0 | 1.0 | 3.0 |
| 2.5 | 2.0 | 2.5 | 1.0 | 1.0 | 3.0 |
| 4.0 | 2.0 | 3.0 | 3.0 | 2.0 | 3.0 |
| 5.0 | 2.5 | 3.0 | 1.0 | 1.0 | 3.0 |

The degree of difference scale is as follows:
1=very similar, 2=slight difference, 3=moderate difference, 4=moderate/large difference and 5=large difference/no fragrance

### EXAMPLE 16

### Evaluation of Bactericidal and Fungicidal Activity of Compositions of Example 4 with Betadine at 1% Total Iodine

Following solutions were evaluated for antimicrobial and antifungal activities.

| | |
|---|---|
| 1% Betadine commercial solution (iodine) | Solution A |
| 1% Iodine, 9% Agrimax 3, 90% water | Solution B |
| (Composition of Example 4A) | |

Iodine use levels in these stock solutions tested were 0.5%, 0.25%, 0.12%, 0.06%, 0.03%, 0.015% and 0.007%, by serial dilution in water.

Solution A was ested at 0.5% iodine, 0.25% iodine, 0.12% iodine, 0.06% iodine, 0.03% iodine, 0.015% iodine and 0.007% iodine by serial dilution.

| | | |
|---|---|---|
| Solution B was tested at: | .5% iodine | 4.5% Agrimax |
| | .25% iodine | 2.3% Agrimax |
| | .125% iodine | 1.2% Agrimax |
| | .06% iodine | 0.6% Agrimax |
| | .03% iodine | 0.3% Agrimax |
| | .015% iodine | 0.15% Agrimax |

Test Organisms

BACTERIA

| | |
|---|---|
| Staphylococcus aureus 6538 | 3.5 × 10⁵ cfu/ml test solution |
| Escherichia cole 9739 | 7.6 × 10⁵ cfu/ml test solution |
| Pseudomonas aeruginosa 9027 | 2.7 × 10⁵ cfu/ml test solution |
| Burkholderia cepacia 25416 | 3.2 × 10⁵ cfu/ml test solution |

MOLD

| | |
|---|---|
| Aspergillus niger 16404 | 9.0 × 10⁴ cfu/ml test solution |
| Chaetomium globosum | 3.0 × 10⁴ cfu/ml test solution |
| Cladosporium species | 2.4 × 10⁴ cfu/ml test solution |
| Paecilomyces species | 1.2 × 10⁴ cfu/ml test solution |

Test Method (MLM 100-7-1)
Summary—An MIC is a serial dilution procedure whereby the test solution is diluted to various concentrations in dilute (50%) Trypticase soy broth and then inoculated with the test organisms. Following incubation, the TSB is examined for turbidity (growth) and the lowest effective concentration is determined, the MIC. This is a measure of static activity. The clear tubes (no growth) are then transferred to a broth with neutralizers, Letheen broth, and reincubated. The tubes are read again and a cidal activity titer is measured.
Result
Bacteria

Iodine in Agrimax 3 had better activity that iodine (Betadine) alone against Staph aureus. With the other organisms, there was no significant difference between iodine (A) and iodine/Agrimax 3 (B).

Mold

In reviewing the data for the 5 species of mold screened, it shows that Betadine is least effective. The antifungal activity for Agrimax 3/iodine is superior to Betadine judging from the MIC (minimum inhibition concentration) and cidal values (See Table below).

TABLE
MIC TEST: IODINE/AGRIMAX VS. IODINE ALONE

| Test Organism | Product | Static Conc | Cidal Conc |
|---|---|---|---|
| Staph aureus (B) | A | .25 | .25 |
| | B | .06 | .06 |
| E. coli (B) | A | .25 | .25 |
| | B | .12 | .12 |
| Pseudomonas | A | .12 | .12 |
| aeruginosa (B) | B | .12 | .12 |
| B. cepacia (B) | A | .25 | .25 |
| | B | .12 | .12 |
| Aspergillus | A | .25 | .25 |
| niger (M) | B | .06 | .06 |
| Aspergillus | A | .12 | .12 |
| fumigatus (M) | B | .015 | .03 |
| Chaetomium | A | .12 | .12 |
| globosum (M) | B | .015 | .015 |
| Chladosporium sp. (M) | A | .12 | .12 |
| | B | .015 | .015 |
| Paecilomyces sp. (M) | A | .12 | .12 |
| | B | .015 | .015 |

Product Codes:
A) 1% Betadine solution (iodine)
B) 1% Iodine, 9% Agrimax, 90% Water (Example 4A)

### EXAMPLE 17

### Evaluation of Fungicidal Efficacy of Composition of Example 9 (5% Triclosan) on Gypsum Boards

The compositions of Example 9 were diluted to 1,000 ppm of triclosan. Then both sides of a gypsum board (cream and grey) sample (2×2×½ in.) were brush coated with each formulation and allowed to dry for 24 hours. The Controls were gypsum boards treated with only water or with only 1,000 ppm of triclosan in dimethyl sulfamide. The treated gypsum boards were then placed on a Petri dish and water was added to saturate the board. The water saturated gypsum boards were then inoculated with a mixed fungal inoculum (Aspergillus niger, Penicillium funiculosum and Stachybotrys chartarum) containing ca. 10⁵ spores/ml. The samples were incubated at 28° C., 80% RH for 30-45 days and rated for the presence or absence of fungal growth thereon. Samples showing no growth were re-inoculated and incubated for another 30-45 days. The results are shown in Table 3 below.

TABLE 3
Presence (+)/Absence (−) of Fungal Growth
on the Surface of the Gypsum Board after Treatment

| Formulation | Cream Side | Grey Side |
|---|---|---|
| Control (water) | + | + |
| Triclosan (1,000 ppm) | + | + |
| AM-1 + triclosan (1,000 ppm) (Ex. 9) | − | − |

The data shows that treatment of both sides of a gypsum board with the Composition of Ex. 9 diluted to contain 1,000 ppm of triclosan inhibited fungal growth whereas the control samples had significant fungal growth.

### EXAMPLE 18

### Biological Activity of Example 11 in Paint

The biological activity of the composition of Example 11 was evaluated on polyvinyl acrylic paint. The microbiological test was based on ASTM D5590-94 (determining the resistance of paint film and related coatings to fungal defacement by accelerated four week agar plate assays).

A paint sample was prepared containing 0.1% by wt. of the formulation of Ex. 11. A standard paint was used as the control. Each paint sample was brush coated onto strips of drawdown paperboard with each sample in duplicate. The strips were air dried for 24 hours. One strip from each sample was then leached with distilled water in a one-gallon container at a flow rate of six changes per day for 24 hours and dried again, while the other strip remained unleached. The strips were cut into 1⅛ inch squares and placed on the surface of solidified malt agar plates.

One square from each sample thus prepared was then inoculated with 1 ml of a mixed spore suspension of Aspergillus niger (ATCC 6275) and Penicillium funiculosum (ATCC 11797) and another square inoculated with a homogenate of Aureobasidium pullulans (ATCC 9348). Each spore suspension contained approximately 10⁶ spores/ml.

All plates were incubated at 28° C. under 85-90% RH for 4 weeks. Observations of growth were recorded weekly. Growth inhibition on the painted sample was recorded based on a scale of “0” to “10” were “0” corresponds to 100% inhibition and “10” corresponds to 0% growth inhibition. The results are shown in Table 4 below.

TABLE 4

| | % | Leaching | Mixed culture | | | | A. pullulans | | | |
|---|---|---|---|---|---|---|---|---|---|---|
| | IPBC | Times | Weeks | | | | Weeks | | | |
| Sample | by wt. | (hours) | 1 | 2 | 3 | 4 | 1 | 2 | 3 | 4 |
| Control | — | 24 | 2 | 2 | 4 | 8 | 2 | 2 | 5 | 7 |
| | | 24 | 3 | 3 | 7 | 8 | 3 | 3 | 7 | 9 |
| 10% IPBC/ | 1.0 | 24 | 0 | 0 | 2 | 3 | 0 | 0 | 0 | 0 |
| AM-1 | | 24 | 0 | 0 | 2 | 3 | 0 | 0 | 1 | 1 |

The data demonstrates that the addition of the compositions of Ex. 11 (1.0% IPBC by wt.) results in significant fungal growth inhibition in a paint formulation compared to an untreated control sample. Growth inhibition is also observed after leaching the paint sample for 24 hours.

### EXAMPLE 19

### Biological Activity of Example 11 in a Stain Formulation on Wood

The biological activity of the composition of Ex. 11 was determined by testing the formulation on a water based stain. The microbiological evaluation described in Example 18 was followed except that the stain was brush coated into wooden blocks and that samples where challenged only with the mixed culture.

Growth inhibition on the painted wood sample was recorded based on a scale of “0” to “10” where “0” corresponds to 100% inhibition and “10” corresponds to 0% inhibition. A zone of inhibition around the sample was measured in millimeters and recorded as Zx (where x represents the zone of inhibition in mm. measured from the edge of the sample). The results indicating efficacy of the composition of Ex. 11 in a water based stain formulation is shown in Table 5.

TABLE 5

| | Mixed Inoculum⁽²⁾ | | | | | | | |
|---|---|---|---|---|---|---|---|---|
| | Leached | | | | | | | |
| | (24 hours) | | | | UnLeached | | | |
| | Weeks | | | | | | | |
| Samples | 1 | 2 | 3 | 4 | 1 | 2 | 3 | 4 |
| Control No Biocide | 6 | 8 | 9 | 10 | 6 | 8 | 9 | 10 |
| w/ 0.50 wt % Agrimax 3/IPBC | Z2 | Z2 | Z2 | 0 | Z5 | Z5 | Z5 | Z3 |

The data demonstrates that the addition of Ex. 11 (0.5% by wt.) results in substantial fungal growth inhibition in a stain when compared to the untreated control.

While the invention has been described with particular reference to certain embodiments thereof, it will be understood that changes and modifications may be made which are within the skill of the art. Accordingly, it is intended to be bound only by the following claims, in which:

1. A stable microemulsion concentrate consisting of, by wt.
(a) 0.1-20% of a bioactive ingredient which is a halogen, triclosan, iodopropargyl butyl carbamate, a quaternary ammonium compound, an organometallic, iodophor, a nitrogen compound, an organo-sulfur compound, chloroisocyanurate, glutaraldehyde or pine oil, a fragrance or body wash, and
(b) 80-99.9% of a polymeric matrix including an alkylated vinyl pyrrolidone copolymer.

2. A stable microemulsion concentrate according to claim 1 consisting of:
(a) 0.5-10% of said bioactive ingredient, and
(b) 90-99.5% of a polymeric matrix comprising
(i) a C₈-C₁₈ alkyl pyrrolidone,
(ii) an emulsifier, and
(iii) an aqueous alkylated vinyl pyrrolidone copolymer.

3. A concentrate according to claim 2 wherein (b) (ii) is an anionic or cationic emulsifier, or a quaternary ammonium salt.

4. A concentrate according to claim 3 wherein (b) (ii) is an anionic emulsifier.

5. A concentrate according to claim 2 wherein (b) (iii) is a C₁₆ alkylated vinyl pyrrolidone copolymer.

6. (canceled)

7. A stable microemulsion concentrate according to claim 1 wherein (a) is iodine, triclosan or iodopropargyl butyl carbamate.

8. A stable microemulsion composition comprising the microemulsion concentrate of claim 2 and water of dilution.

9. A stable microemulsion composition of claim 8 which is an effective bactericide and/or fungicide.

10. A method of treating a disease which comprises applying the stable microemulsion composition of claim 8 thereto.
